(19) United States
(12) Patent Application Publication
(10) Pub. No.: US 2012/0109142 A1
(43) Pub. Date: May 3, 2012

(54) Surgical Screwdriver

(76) Inventor: Alan Dayan, Brooklyn, NY (US)

(21) Appl. No.: 12/912,972

(22) Filed: Oct. 27, 2010

(51) Int. Cl. A61B 17/88; B25B 15/04

(52) U.S. Cl. 606/104; 81/60; 81/450

(57) ABSTRACT

Disclosed is a surgical screwdriver providing a secure fit for a fastener for use in inserting the fastener into a patient. The device creates negative pressure within the tip of the surgical screwdriver for holding the fastener in place. Negative pressure is created by drawing air through a nozzle at the tip of the device. Air is pulled through the device and out the proximal end of the device's handle via suction tubing connected to the suction attachment of the proximal end of the handle.

### TECHNICAL FIELD

The present application is directed to surgical screwdrivers for inserting a fastener into a patient and, more particularly, the application relates to screwdrivers and methods of use for securing the fastener to the screwdriver.

### BACKGROUND

Various surgical procedures utilize one or more fasteners that are attached within the patient. The fasteners include a shaft that is inserted into a support member such as an implant, bone, or tissue. The fastener further includes a head positioned at an end of the shaft that includes a receiver for engagement with a screwdriver. The shaft may include threads to assist in inserting the screw into the support member and in preventing the screw from backing out of the support member. The head may include a variety of different receivers with different shapes and sizes depending upon the context of use.

The screwdriver includes a tip that engages the receiver and a shaft to rotate the fastener and drive it into the support member. The screwdriver may further include a handle that is grasped and rotated by the surgeon to insert the fastener into the support member.

A specific use for such cementless acetabular components, include those for use during total hip arthroplasty. Two of the most critical factors for success are initial implant stability and intimate contact of the implant with the host bone. To facilitate these two criteria for bony ingrowth onto the acetabular component many models have incorporated screw holes in the implant dome for the placement of adjunctive fixation. In addition to augmenting initial implant stability. The use of screws in the posterosuperior quadrant of an acetabular component serves to pull the ingrowth surface closer to the host bone when fully tightened.

The technical difficulty of inserting acetabular screws during total hip arthroplasty is partly due to the deep operative exposure that the screwdriver and screw complex need to be inserted into and the angle which needs to obtained to match the corresponding screw hole in the implant. Current screwdriver models allow for manually altering the angle of the screw head-screwdriver coupling with respect to the shaft However it is not uncommon for the coupling to disengage and the screw fall into the acetabular component. This lack of a secure fit between the screw head and the screwdriver can make this aspect of the procedure tedious and time-consuming.

Therefore what is needed is a device and method capable of providing a secure fit between the screw head and the screwdriver in a surgical setting.

### SUMMARY

The present invention comprises a device directed to a surgical screwdriver providing a secure fit for a fastener for use in inserting the fastener into a patient. The device creates negative pressure within the tip of the surgical screwdriver for holding the fastener in place. Negative pressure is created by drawing air through a nozzle at the tip of the device to create suction for holding the fastener in place. Air is pulled through the device and out the proximal end of the device's handle via suction tubing connected to the suction attachment of the proximal end of the handle.

In greater detail, the surgical device to drive a medical fastener within a patient comprises a handle and an elongated shaft connected to the handle. The shaft includes a proximal end and a distal end, wherein the distal end includes a tip sized to engage the fastener. A cavity is defined within the handle which extends through the length of the handle. A hollow chamber is formed within and defined by the shaft and in is in fluid communication with the cavity. The hollow chamber formed within the shaft extends out from the cavity at the distal end of the handle to a nozzle formed within the tip whereby air can travel there through. Negative pressure is created by drawing or urging air through the nozzle for retaining the fastener within the tip.

A further embodiment includes a surgical device to drive a medical fastener within a patient including a handle and elongated shaft connected to the handle. A tip having a nozzle formed within is located at the distal end of the shaft and is connected to a hollow chamber for drawing in air to create negative pressure. The device further includes a joint connector connecting a first and second portion of the shaft wherein the joint connector is positioned before the tip whereby the device can be orientated with a screw hole. A ratchet is also included which is operatively positioned within the device whereby the device is capable to progressively tightening the fastener without requiring 360 degrees of rotation.

An additional embodiment includes the surgical device further including a flexible conduit housed within the hollow chamber of the shaft and extending from the proximal end to the distal end of the shaft and operatively connected to the nozzle. The flexible conduit may taper from the proximal end to the distal end of the shaft. Additionally, a suction attachment at the proximal end of the handle is included and may be tapered for ease of attachment. The device may further include a trigger, integrated with the handle, and having a first state in which the flow of air through the cavity within the handle is blocked and a second state in which the flow of air is allowed to flow through the cavity within the handle to create a negative pressure at the tip. When the flow of air is blocked the fastener is released and when air is allowed to flow the fastener becomes attached to the tip.

### DRAWINGS

In the drawings:

FIG. 1 depicts the surgical device for driving a medical fastener showing a channel within both the handle and the shaft whereby air is drawn through the nozzle portion to create a negative pressure to hold the fastener in place;

FIG. 2 illustrates an embodiment of the joint connector for directing the tip and fastener to the screw hole, a ball joint is illustrated within this figure;

FIG. 3 shows the tip and the fastener; and

FIG. 4 depicts the hinge joint for the joint connector.

### DETAILED DESCRIPTION

Disclosed is a surgical screwdriver providing a secure fit for a fastener for use in inserting the fastener into a patient. The device creates negative pressure within the tip of the surgical screwdriver for holding the fastener in place. Negative pressure is created by drawing air through the nozzle at the tip of the device resulting in adhesion between the fastener and the tip. Air is pulled through the device and out the proximal end of the device's handle via suction tubing connected to the suction attachment of the proximal end of the handle.

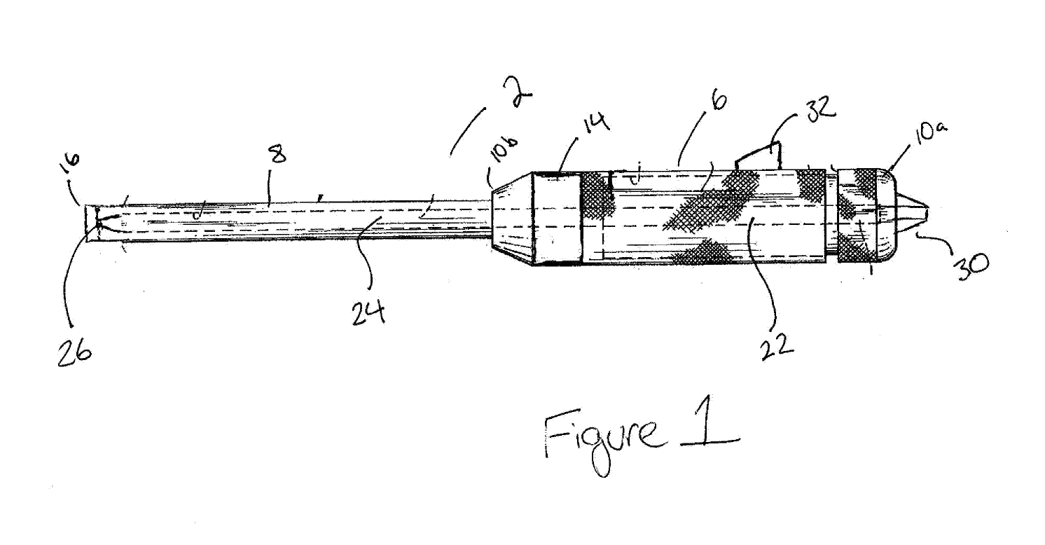

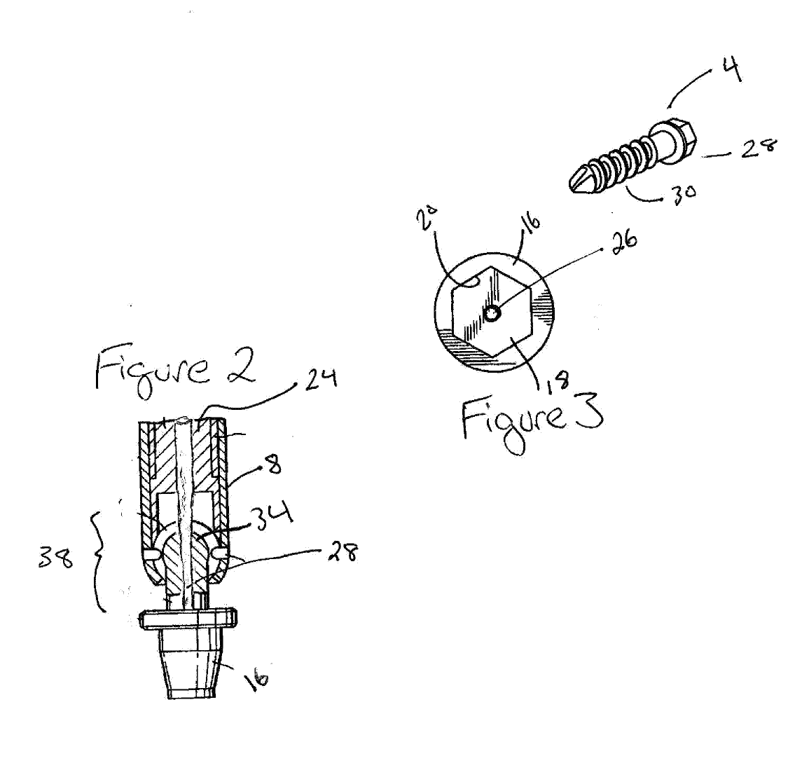

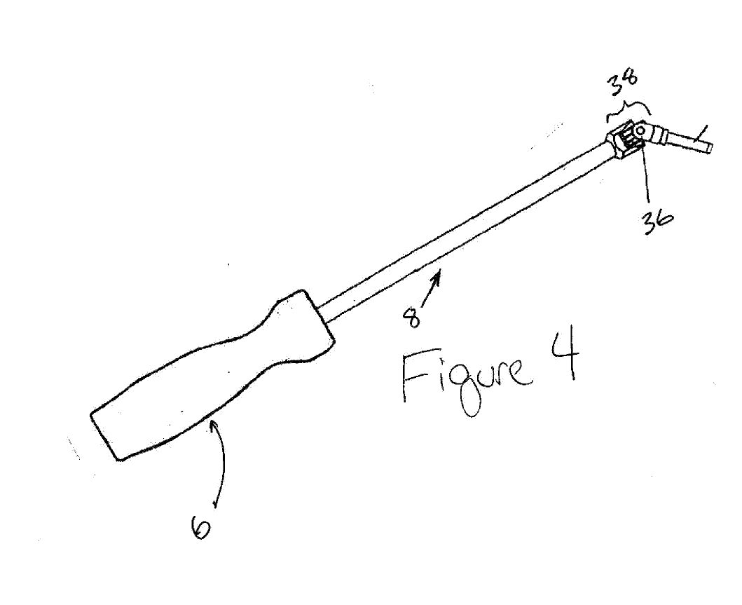

Referring now in greater detail to the drawings in which like numerals indicate like items throughout the several views, FIGS. 1-4 depict the present device for securing a fastener in a surgical screwdriver for inserting the fastener into a patient, in the various embodiments of the present invention.

Turning now to the FIGS. 1-4, the present apparatus is shown in various perspective views. The present surgical screwdriver 2 is designed in to drive a medical fastener 4 within a patient. The screwdriver 2 includes a handle 6 and an elongated shaft 8 connected to the handle 6. The shaft 8 may run the entire length of the handle 6 and may be housed within the handle 6 such that from the proximal 10a to the distal end 10b of the handle 6 the shaft resides 8.

In greater detail, the handle 6 provides a surface for grasping and manipulating the screwdriver 2. The handle 6 is grasped by the surgeon and rotated to apply torque to the fastener 4. In one embodiment a ratchet assembly 14 is operative positioned within the screwdriver 2 whereby the screwdriver 2 is capable of progressively tightening the fastener 4 without requiring 360 degrees of rotation. The screwdriver 2 is not limited to any one type of ratchet assembly 14 and such assemblies would be readily understood and known to those skilled in the art.

Additionally, the handle 6 may also be used to apply a downward force to further facilitate insertion of the fastener 4. In one embodiment, handle 6 is positioned on a proximal end of the shaft 8. In another embodiment, handle 6 is positioned along a central section of the shaft 8. A further embodiment may include a plurality of handles 6 positioned along the shaft 8. The handle 6 may include varying widths having relatively wide and narrow sections. The handle 6 may further include an ergonomic shape. In one embodiment, handle 6 includes a textured surface to prevent slippage. The handle 6 may also be removeably attached to the shaft 8. Additionally, it is contemplated that no handle may be necessary in an embodiment, for example when the shaft 8 is connected to a powered rotational device requiring no handle 6.

The fastener 4 may have a variety of shapes and sizes. In one embodiment, the fastener 4 includes a hex head 28 and an elongated portion 30. In an embodiment, fastener 4 is a screw with the head 28 being shaped and sized to engage with the tip 16 and the elongated portion 30 including helical threads. The fastener 4 may be constructed of a resorbable material comprising polymers and/or co-polymers made from lactic acid and/or glycolic acid. The fastener 4 may also be constructed of metal.

The shaft 8 includes a proximal end and a distal end, wherein the distal end includes a tip 16 sized to engage the fastener 4. Shaft 8 may have a variety of lengths depending upon the context of use. In one embodiment, shaft 8 has a length so that the tip 16 may be positioned within the patient with the handle 6 exterior to the patient. Shaft 8 may further have a variety of cross-sectional shapes including circular, rectangular, polygonal, and formed from most any material. Typically the handle is formed from a material applicable for sterilization.

In one embodiment, the tip 16 is integral with the shaft 8. In another embodiment the tip 16 is a separate member that attaches to the shaft 30. The tip 16 may include a distal edge 20 may extend outward having a variety of lengths. The tip 16 of the screwdriver 2 may extend outward from the shaft 8 with the distal edge 20 configured to engage the fastener 4. As illustrated, the distal edge 20 is shaped to engage with a hex head fastener 4. Additional shapes may be available to engage the fastener 4 and include but not limited to Allen, SupaDriv, TORX, PoziDriv, and Robertson.

A cavity 22 is defined within the handle 6 which extends through the length of the handle 6. The cavity 22 may also house the shaft 8 in one embodiment wherein the hollow chamber 24 is formed within the shaft 8 and runs the length of the handle 6. The hollow chamber 24 is defined by the shaft 8 is in fluid communication with the cavity 22. The hollow chamber 24 formed within the shaft 8 in an embodiment extends out from the cavity 22 at the distal end 10b of the handle 6 to a nozzle 26 formed within the tip 16. Negative pressure is created by drawing or urging air through the nozzle 26 for retaining the fastener 4 within the tip 16.

The screwdriver 2 may further include a flexible conduit 28 housed within the hollow chamber 24 of the shaft 8 and extending from the proximal end to the distal end of the shaft 8 and operatively connected to the nozzle 26. The flexible conduit 28 may taper from the proximal end to the distal end of the shaft 8. Additionally, a suction attachment 30 at the proximal end 10a of the handle 6 may be tapered. The screwdriver 2 may further include a trigger 32, integrated with the handle, and having a first state in which the flow of air through the cavity within the handle is blocked and a second state in which the flow of air is allowed to flow through the cavity within the handle 6.

Furthermore, in an additional embodiment a joint connector 38 connecting a first and second portion of the shaft 8 wherein the joint connector 38 is positioned before the tip 16 whereby the device can be orientated with a screw hole. The joint connecter 38 may be, by way of example and not limitation, a ball joint 34 or a hinge joint 36.

In the use of the present method, screw holes are made prior to the implantation of an acetabular component and an appropriate length of fastener 4 is chosen. The appropriate fastener 4 is then inserted into the tip 16 of the screwdriver 2. Suction tubing may be attached to the screwdriver 2 before or after the insertion of the fastener 4. The negative pressure created by the air being drawn into the nozzle holds the fastener 4 in place. The angle of the tip may be oriented as needed to place the fastener 4 toward the screw hole. Once inserted, the fastener 4 may be tightened using the screwdriver 2. In an additional embodiment the fastener 4 may be tightened by the screwdriver 2 having a ratchet 14. Once the fastener 4 is firmly seated within the screw hole the negative pressure may be relieved by removing the tubing or using the switch 32 either housed within the handle 6 or the shaft 8.

While specific embodiments have been described in detail in the foregoing detailed description and illustrated in the accompanying drawings, those with ordinary skill in the art will appreciate that various modifications and alternatives to those details could be developed in light of the overall teachings of the disclosure. Accordingly, the particular arrangements disclosed are meant to be illustrative only and not limiting as to the scope of the invention, which is to be given the full breadth of any claims that are derivable from the description herein, and any and all equivalents thereof.

What is claimed is:

1. A surgical device to drive a medical fastener within a patient comprising:
a handle;
an elongated shaft connected to the handle, the shaft including a proximal end and a distal end;
a tip at the distal end sized to engage the fastener and including a nozzle;
a cavity defined within the handle extending through the handle; and
a hollow chamber defined within the shaft and in communication with the cavity, the hollow chamber extending outwardly from the cavity and to the nozzle in the tip whereby the negative pressure can be applied to the tip for retaining the fastener by drawing air through the chamber.

2. The device of claim 1, further including a ratchet whereby the device is capable of progressively tightening the fastener without requiring 360 degrees of rotation.

3. The device of claim 2, wherein the ratchet resides within the handle portion of the device.

4. The device of claim 1, further including a trigger, integrated with the handle, having a first state in which the flow of air through the cavity within the handle is blocked and a second state in which the flow of air is allowed to flow through the cavity within the handle.

5. The device of claim 1, further including a joint connector connecting a first and second portion of the shaft wherein the joint connector is positioned before the tip whereby the device can be orientated with a screw hole.

6. The device of claim 5, wherein the joint connector is selected from the group consisting of a ball joint, a hinge joint and combinations thereof.

7. The device of claim 1, further including a flexible conduit housed within the hollow chamber of the shaft and extending from the proximal end to the distal end of the shaft.

8. The device of claim 8, wherein the flexible conduit tapers from the proximal end to the distal end of the shaft.

9. The device of claim 1, further including a suction attachment at the proximal end of the handle.

10. A surgical device to drive a medical fastener within a patient comprising:
a handle;
an elongated shaft connected to the handle, the shaft including a proximal end and a distal end;
a tip at the distal end sized to engage the fastener;
a cavity defined within the handle extending through the handle and including a nozzle;
a hollow chamber defined within the shaft and in communication with the cavity, the hollow chamber extending outwardly from the cavity and to the nozzle in the tip whereby negative pressure can be applied to the tip for retaining the fastener by drawing air through the chamber;
a joint connector connecting a first and second portion of the shaft wherein the joint connector is positioned before the tip whereby the device can be orientated with a screw hole; and
a ratchet operatively positioned within the device whereby the device is capable of progressively tightening the fastener without requiring 360 degrees of rotation.

11. The device of claim 10, wherein the ratchet resides within the handle portion of the device.

12. The device of claim 10, further including a trigger, integrated within the handle, having a first state in which the flow of air through the cavity within the handle is blocked and a second state in which the flow of air is allowed to flow through the cavity within the handle.

13. The device of claim 10, wherein the joint connector is selected from the group consisting of a ball joint, hinge joint and combinations thereof.

14. The device of claim 10, further including a flexible conduit housed within the hollow chamber of the shaft and extending from the proximal end to the distal end of the shaft.

15. The device of claim 14, wherein the flexible conduit tapers from the proximal end to the distal end of the shaft.

16. The device of claim 10, further including a suction attachment at the proximal end of the handle.

17. A surgical device to drive a medical fastener within a patient comprising:
a handle;
an elongated shaft connected to the handle, the shaft including a proximal end and a distal end;
a tip at the distal end sized to engage the fastener;
a cavity defined within the handle extending through the handle and including a nozzle;
a hollow chamber defined within the shaft and in communication with the cavity, the hollow chamber extending outwardly from the cavity to the nozzle in the tip whereby the negative pressure can be applied to the tip for retaining the fastener by drawing air through the chamber;
a joint connector connecting a first and second portion of the shaft wherein the joint connector is positioned before the tip whereby the device can be orientated with a screw hole;
a ratchet operative positioned within the device;
a suction attachment at the proximal end of the handle; and
a flexible conduit housed within the hollow chamber of the shaft and extending from the proximal end to the distal end of the shaft.

18. The device of claim 17, wherein the flexible conduit tapers from the proximal end to the distal end of the shaft.

19. The device of claim 17, wherein the suction attachment at the proximal end of the handle is tapered.

20. The device of claim 17, further including a trigger, integrated with the handle, having a first state in which the flow of air through the cavity within the handle is blocked and a second state in which the flow of air is allowed to flow through the cavity within the handle.
